SEQUENCE LISTING (3 sequences)

SEQ ID NO: 1 - protein, 1068 aa, synthetic construct
QYDSYDVKSGVAVGGLAGYPGPAGPPGPPGPPGTSGHPGSPGSPGYQGPPGEPGQAGPSGPPGPPGAIGPSGPAGKDGESGERGFPGERGVQGPPGIKGPAGIPGFPGMKGHRGFDGRNGEKGETGERGFPGERGFPGERGVQGPMGERGFPGERGVQGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGERGFPGERGVQGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGERGFPGERGVQGLAGAPGLRGGAGPPGPEGGKGAAGPPGPPGAAGTPGERGFPGERGVQGSPGPKGDKGEPGGPGADGVPGKDGPRGPTGPIGPPGPAGQPGDKGEGGAPGLPGIAGPRGSPGERGETGPPGPAGFPGAPGQNGERGFPGERGVQGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGERGFPGERGVQGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYG

SEQ ID NO: 2 - DNA, 3204 bp, synthetic construct
  source 1..3204: mol_type = other DNA
caatacgactcttacgatgtcaaatctggagtggctgttggtggattggctggataccctggaccagctggcccaccaggtccacctggacctccaggcacttctggacatccaggttcccctggatctccaggttaccaaggtccaccaggtgaaccaggtcaagcaggtccaagtggtccaccaggtccacctggtgctatcggtccatccggacctgccggtaaagacggtgaatctggcgaaagaggtttccctggtgaacgtggcgtacagggaccacctggcatcaagggacctgctggcatccctggatttccaggcatgaagggtcatcgaggctttgatggcagaaatggcgagaaaggtgaaacgggagaaagaggctttccaggcgaaaggggattccctggcgagcgtggagttcagggacctatgggtgagagaggctttccaggtgaaagaggcgtacaaggtttgccaggtgctgctggagcaagaggtaacgatggagcacgaggcagtgatggccaacctggtccacctggtccacctggtacggcaggtttccctggtagtccaggcgctaaaggtgaagtgggtccagctggttctccaggtagtaatggcgcaccaggtcagagaggtgagcctggacctcaaggtcacgctggagcacagggtccacctggtccacctggtatcaacggttctccaggcggcaaaggtgagatgggacctgctggtatcccaggtgctccaggtctaatgggtgccagaggtccacctggtccagctggagctaatggagcaccaggcctaagaggtggagcaggcgaacctggaaagaatggagctaagggagagcctggccctagaggtgagcgtggtgaagctggaatccctggtgttccaggcgccaaaggcgaagatggtaaagacggaagtccaggtgagccaggtgccaacggagaacgaggctttccaggcgagaggggagttcaaggctttagaggtccagccggacctaatggcatacctggtgagaagggaccagctggtgagagaggtgcacctggtccagccggaccaagaggtgctgccggagaacctggcagagatggagttccaggtggcccaggtatgagaggtatgcctggatcaccaggcggtccaggttctgatggtaagcctggtccacctggatctcagggagaaagtggtagaccaggtccacctggaccatccggtccaagaggtcaaccaggtgtgatgggtttccctggacctaaaggcaacgacggtgcacctggtaagaacggtgaaagaggtggtccaggcggacctggaccacaaggtccaccaggcaagaacggagagactggaccacagggacctccaggaccaaccggaccaggcggagacaaaggtgacactggtccacctggacctcagggattgcagggtttaccaggcactggcggtccacctggcgagaacggtaaacctggcgaaccaggccctaagggtgatgctggtgcacctggagcccctggtggcaaaggcgagagaggtttccctggagaacgaggtgttcagggtttagccggagcaccaggcttgagaggcggtgccggaccaccaggtccagagggtggtaaaggcgctgccggaccacctggaccaccaggcgctgctggtacaccaggcgaacgtggatttccaggtgaacgtggagtacaaggtagtccaggtccaaagggagacaagggtgaacctggtggccctggtgctgatggtgtgcctggcaaagatggtccacgtggtccaactggacctattggtccacctggtccagcaggtcagcctggagataagggcgaaggcggtgctccaggcttgcctggcatcgctggaccacgaggttcacctggagagagaggcgaaacgggaccaccaggtccagcaggcttccctggagcacctggtcagaatggtgaacgaggtttcccaggtgagagaggtgtacagggagagaagggagagggtggacctccaggcgtagctggtccacctggaggttccggaccagccggtccaccaggtccacaaggtgtgaaaggcgaacgaggctcacctggaggcccaggtgcagctggatttccaggtgctagaggcttgccaggtccaccaggctctaacggaaaccctggtccacctggaccttctggctctccaggcaaagacggacctccaggtccagcaggtaacactggcgctccaggttcaccaggtgtctctggtccaaagggtgacgctggccaaccaggcgagaagggaagtccaggcgcacaaggccctccaggcgcacctggtccattaggtatcgcaggtatcacgggtgcaagaggtttggctggtccacctggaatgcctggaccaagaggttctccaggtccacagggagttaaaggcgaatcaggtaagccaggtgcaaacggcttatctggagaaagaggtccacctggtccacaaggccttccaggccttgcaggtacagccggagagccaggtagagatggtaaccctggctcagatggactgccaggtagggatggatcacctggcggaaagggcgacagaggtgagaatggctcccctggtgcacctggtgcacctggccatccaggacctccaggccctgttggcgaaagaggcttcccaggcgaacgtggtgttcaaggccctgccggtccagctggcgcacctggaccagcaggcagtagaggcgcacctggacctcaaggcccaagaggcgataagggtgaaactggtgagcgaggcgcagctggcattaaaggccatcgtggctttccaggcaatccaggcgctccaggcagtccaggtccagctggtcaacaaggtgctattggctcaccaggtccagccggacctcgtggacctgtaggtccatctggtccaccaggtaaagatggcacttcaggtcatccaggtccaatcggtccacctggacctagaggcaacagaggcgagcgaggttctgaaggctctccaggtcaccctggacaaccaggtccacctggtccacctggcgcacctggaccatgttgtggaggtgtgggagctgctgctatcgctggaataggcggagagaaagctggtggtttcgcaccttattacggt

SEQ ID NO: 3 - DNA, 9276 bp, synthetic construct
  source 1..9276: mol_type = other DNA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattccctagggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

(19) United States
(12) Patent Application Publication
(10) Pub. No.: US 2026/0083883 A1
(43) Pub. Date: Mar. 26, 2026

(54) RECOMBINANT HUMAN TYPE III COLLAGEN FOR PROMOTING ANGIOGENESIS, AND PREPARATION METHOD AND APPLICATION METHOD THEREOF

(71) Applicant: Northwest University, Xi'an (CN)

(72) Inventors: Weina Li, Xi'an (CN); Yaqian Zhang, Xi'an (CN); Daidi Fan, Xi'an (CN); Xinyue Deng, Xi'an (CN); Yanting Yuan, Xi'an (CN); Qiyi Jiang, Xi'an (CN); Ziyang Niu, Xi'an (CN); Jingwen Zhai, Xi'an (CN); Linmiao Song, Xi'an (CN); Min Yang, Xi'an (CN); Zhe Wan, Xi'an (CN); Xinzheng Fu, Xi'an (CN); Tingyuan Xiao, Xi'an (CN)

(21) Appl. No.: 19/035,974

(22) Filed: Jan. 24, 2025

(30) Foreign Application Priority Data
Sep. 26, 2024 (CN) 2024113497035

(51) Int. Cl. A61L 31/04; A61L 27/24; A61L 27/50; C07K 1/36; C07K 14/78; C12N 1/16; C12N 15/81

(52) U.S. Cl. CPC A61L 31/044; A61L 27/24; A61L 27/507; C07K 1/36; C07K 14/78; C12N 1/165; C12N 15/815; A61L 2430/34

(57) ABSTRACT

Recombinant human type III collagen for promoting angiogenesis, and a preparation method and an application method thereof are provided, which relate to the field of genetic engineering technologies. The amino acid of the recombinant human type III collagen is as shown in SEQ ID NO: 1. The method replaces nine different sites of a full-length natural type III collagen with a GFOGER sequence from type I collagen specifically binding to integrin sites, thereby enhancing promote angiogenic function without changing a total length of natural full-length amino acids. Meanwhile, the method replaces four natural integrin sites with lower adhesion rates, thus the recombinant type III collagen have higher adhesion, to further promote angiogenesis. Through this modification method, the angiogenesis rate will be increased by more than 37%, and the molecular weight of protein translated by the recombinant human type III collagen is moderate, making it easy to prepare.

### CROSS-REFERENCE TO RELATED APPLICATION

This application claims priority to Chinese Patent Application No. 202411349703.5, filed on Sep. 26, 2024, which is herein incorporated by reference in its entirety.

### TECHNICAL FIELD

The disclosure relates to the field of genetic engineering technologies, and more particularly to recombinant human type III collagen for promoting angiogenesis, and a preparation method and an application method thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25005TBYX-USP1-SL.xml. The XML file is 17,241 bytes; is created on Jan. 20, 2025; and is being submitted electronically via patent center.

### BACKGROUND

Collagen is one of the most important and abundant proteins in mammals, and is a structural protein found in the skin, connective tissue, bones, and other tissues of the human body. A content of the collagen in the human body is about 30% of total protein. The collagen is a structural protein, and is a main component of extracellular matrix. Type III collagen consists of three peptide chains that curl and twist to the right, forming a triple helix. Analysis of the primary structure indicates that a long segment sequence of a polypeptide chain of the type III collagen is repeated from the glycine (Gly)-X—Y amino acid sequence. Specifically, X is usually proline, Y is usually hydroxyproline and hydroxylysine, and the latter two amino acids are rare in other proteins.

The collagen has low immunogenicity and functions such as promoting tissue repair and hemostasis, and has been widely used in fields such as food, cosmetics, biomedical materials, and pharmaceuticals. At present, the collagen is mainly extracted from animal tissues, but materials derived from the animal tissues are at risk of viral infection, such as mad cow disease (also referred to as bovine spongiform encephalopathy, abbreviated as BSE). At the same time, natural collagen is widely present in skin tissues and blood vessels, and its expression is upregulated during the growth and wound healing process of organisms, but the rate of promoting angiogenesis by the natural collagen is relatively low.

### SUMMARY

A purpose of the disclosure is to provide recombinant human type III collagen for promoting angiogenesis, and a preparation method and an application method thereof, which solves the problem of low angiogenesis rate promoted by natural collagen protein.

The technical solutions adopted by the disclosure are as follows.

Recombinant human type III collagen for promoting angiogenesis is provided, and the amino acid of the recombinant human type III collagen is as shown in SEQ ID NO: 1, and the nucleotide sequence of a gene encoding the recombinant human type III collagen is as shown in SEQ ID NO: 2.

The recombinant human type III collagen is used to prepare a cardiovascular stent material or an artificial blood vessel.

The recombinant human type III collagen is used to prepare a skin tissue repair material.

The recombinant human type III collagen is used to prepare a preparation for treating cardiovascular diseases.

An expression vector is provided, and the expression vector includes the nucleotide sequence as shown in SEQ ID NO: 2.

A host cell is provided, and the host cell includes the above expression vector.

The host cell is any one selected from the group consisting of Pichia pastoris, Saccharomyces cerevisiae, Escherichia coli, and Bacillus subtilis.

A method for preparing the recombinant human type III collagen by using the host cell is provided, including:
cultivating the host cell in a culture medium, and inducing expression of the recombinant human type III collagen to obtain an induced product, and purifying the induced product to obtain the recombinant human type III collagen.

A method for the purifying is any one selected from the group consisting of a salting-out method, an ultrafiltration method, an affinity chromatography method and a gel filtration chromatography method.

The beneficial effects of the disclosure are as follows. The disclosure replaces nine different sites of a full-length natural type III collagen with a glycine-phenylalanine-hydroxyproline-glycine-glucose-arginine (GFOGER) sequence from type I collagen that specifically bind to integrin sites, thereby enhancing promote angiogenic function of the type III collagen without changing the total length of amino acids of the full-length natural type III collagen. At the same time, the disclosure also replaces four natural integrin sites with lower adhesion rates, so that the recombinant human type III collagen have higher adhesion, to further promote angiogenesis. Through this modification method, the angiogenesis rate will be increased by more than 37%, and the molecular weight of the protein translated by the recombinant human type III collagen is moderate, making the recombinant human type III collagen easy to prepare.

The recombinant human type III collagen of the disclosure performs codon optimization to better adapt host bacteria.

The recombinant human type III collagen of the disclosure removes a full-length chain sequence of a C-terminal and a N-terminal of a collagen coding region, which effectively avoids a series of antigen immune reactions.

The recombinant human type III collagen prepared by the disclosure is expressed by Pichia pastoris engineering bacteria. The recombinant human type III collagen has no potential for endotoxins and does not carry a histidine tag. It can be purified through molecular sieves and the target protein (i.e., the recombinant human type III collagen) can be obtained directly without the need for additional removal of a histidine tag sequence.

The recombinant human type III collagen prepared by the method of the disclosure can significantly promote angiogenesis, and be applied in cardiovascular diseases, wound healing and skin repair without causing immune rejection. The recombinant human type III collagen prepared by the method of the disclosure can be widely used in medical materials, cosmetics, and other fields.

The preparation method of the recombinant human type III collagen of the disclosure is suitable for industrial large-scale production, and the prepared product has no animal source of infection, thus having higher biological safety.

### BRIEF DESCRIPTION OF DRAWINGS

FIG. 1 illustrates a schematic diagram of a purification result of recombinant human type III collagen.

FIG. 2 illustrates a schematic diagram of effects of different concentrations of the recombinant human type III collagen on cell proliferation.

FIG. 3 illustrates a schematic diagram of angiogenesis of an embodiment 3; specifically, from left to right are bovine serum albumin (BSA), type III natural collagen, and recombinant human type III collagen.

### DETAILED DESCRIPTION OF EMBODIMENTS

In order to enable those skilled in the art to better understand the technical solution of the disclosure and implement it, the disclosure will be further described in conjunction with specific embodiments and accompanying drawings.

In the descriptions of the disclosure, unless otherwise specified, all reagents used are commercially available and all methods used are conventional techniques in this field.

The inventive concept is as follows. Angiogenesis is related to cell migration and proliferation, and cell adhesion is a prerequisite for the angiogenesis. Extracellular matrix (ECM) is a non-cellular component that exists in all tissues and organs, which is mainly secreted by fibroblasts. The ECM occupies most of the connective tissue area and provides a necessary physical framework for cellular compositions. Collagen is a main component of ECM, and is recognized by receptors such as integrins, discoid domain receptors, and multi ligand proteoglycans, to thereby play an important functional role in controlling key cellular responses such as adhesion, migration, proliferation, differentiation, and survival.

Due to more type III collagen existed in blood vessels, the inventor replaces nine different sites of a full-length natural type III collagen with a GFOGER sequence from type I collagen that specifically binds to integrin sites, thereby enhancing promote angiogenic function of the type III collagen without changing the total length of amino acids of the full-length natural type III collagen. At the same time, the inventor also replaces four natural integrin sites with lower adhesion rates, so the recombinant human type III collagen have higher adhesion, to further promote angiogenesis.

A purpose of the disclosure is to provide recombinant human type III collagen with an ability to promote angiogenesis. The recombinant human type III collagen for promoting angiogenesis can effectively promote angiogenesis, which can be applied in cardiovascular diseases, wound healing and skin repair without causing immune rejection. The recombinant human type III collagen can be widely used in medical materials, cosmetics, and other fields.

The disclosure provides recombinant human type III collagen for promoting angiogenesis, and the amino acid sequence of the recombinant human type III collagen is as shown in SEQ ID NO: 1.

SEQ ID NO: 1 is shown as follows:

QYDSYDVKSGVAVGGLAGYPGPAGPPGPPGPPGTSGHPGSPGSPGYQGPPGEPG
QAGPSGPPGPPGAIGPSGPAGKDGESGERGFPGERGVQGPPGIKGPAGIPGFPGMKGHRG
FDGRNGEKGETGERGFPGERGFPGERGVQGPMGERGFPGERGVQGLPGAAGARGNDG
ARGSDGQPGPPGPPGTAGFPGSPGAKGEVGPAGSPGSNGAPGORGEPGPQGHAGAQGP
PGPPGINGSPGGKGEMGPAGIPGAPGLMGARGPPGPAGANGAPGLRGGAGEPGKNGAK
GEPGPRGERGEAGIPGVPGAKGEDGKDGSPGEPGANGERGFPGERGVQGFRGPAGPNGI
PGEKGPAGERGAPGPAGPRGAAGEPGRDGVPGGPGMRGMPGSPGGPGSDGKPGPPGSQ
GESGRPGPPGPSGPRGQPGVMGFPGPKGNDGAPGKNGERGGPGGPGPQGPPGKNGETG
PQGPPGPTGPGGDKGDTGPPGPQGLQGLPGTGGPPGENGKPGEPGPKGDAGAPGAPGG
KGERGFPGERGVQGLAGAPGLRGGAGPPGPEGGKGAAGPPGPPGAAGTPGERGFPGER
GVQGSPGPKGDKGEPGGPGADGVPGKDGPRGPTGPIGPPGPAGQPGDKGEGGAPGLPGI
AGPRGSPGERGETGPPGPAGFPGAPGQNGERGFPGERGVQGEKGEGGPPGVAGPPGGSG
PAGPPGPQGVKGERGSPGGPGAAGFPGARGLPGPPGSNGNPGPPGPSGSPGKDGPPGPA
GNTGAPGSPGVSGPKGDAGQPGEKGSPGAQGPPGAPGPLGIAGITGARGLAGPPGMPGP
RGSPGPQGVKGESGKPGANGLSGERGPPGPQGLPGLAGTAGEPGRDGNPGSDGLPGRD
GSPGGKGDRGENGSPGAPGAPGHPGPPGPVGERGFPGERGVQGPAGPAGAPGPAGSRG
APGPQGPRGDKGETGERGAAGIKGHRGFPGNPGAPGSPGPAGQQGAIGSPGPAGPRGPV
GPSGPPGKDGTSGHPGPIGPPGPRGNRGERGSEGSPGHPGQPGPPGPPGAPGPCCGGVGA
AAIAGIGGEKAGGFAPYYG.

The disclosure provides recombinant human type III collagen for promoting angiogenesis, which performs codon optimization for expression of a host cell. During designing, two ends of a gene for encoding the recombinant human type III collagen are added with signal peptide cleavage sites, and EcoR I and Not I restriction enzyme sites, to facilitate later gene manipulation. Through the above optimization, the nucleotide sequence of the gene is as shown in SEQ ID NO: 2.

SEQ ID NO: 2 is shown as follows:

CAATACGACTCTTACGATGTCAAATCTGGAGTGGCTGTTGGTGGATTGGCTGG
ATACCCTGGACCAGCTGGCCCACCAGGTCCACCTGGACCTCCAGGCACTTCTGGACA
TCCAGGTTCCCCTGGATCTCCAGGTTACCAAGGTCCACCAGGTGAACCAGGTCAAGC
AGGTCCAAGTGGTCCACCAGGTCCACCTGGTGCTATCGGTCCATCCGGACCTGCCGG
TAAAGACGGTGAATCTGGCGAAAGAGGTTTCCCTGGTGAACGTGGCGTACAGGGAC
CACCTGGCATCAAGGGACCTGCTGGCATCCCTGGATTTCCAGGCATGAAGGGTCATC
GAGGCTTTGATGGCAGAAATGGCGAGAAAGGTGAAACGGGAGAAAGAGGCTTTCC
AGGCGAAAGGGGATTCCCTGGCGAGCGTGGAGTTCAGGGACCTATGGGTGAGAGAG
GCTTTCCAGGTGAAAGAGGCGTACAAGGTTTGCCAGGTGCTGCTGGAGCAAGAGGT
AACGATGGAGCACGAGGCAGTGATGGCCAACCTGGTCCACCTGGTCCACCTGGTAC
GGCAGGTTTCCCTGGTAGTCCAGGCGCTAAAGGTGAAGTGGGTCCAGCTGGTTCTCC
AGGTAGTAATGGCGCACCAGGTCAGAGAGGTGAGCCTGGACCTCAAGGTCACGCTG
GAGCACAGGGTCCACCTGGTCCACCTGGTATCAACGGTTCTCCAGGCGGCAAAGGT
GAGATGGGACCTGCTGGTATCCCAGGTGCTCCAGGTCTAATGGGTGCCAGAGGTCC
ACCTGGTCCAGCTGGAGCTAATGGAGCACCAGGCCTAAGAGGTGGAGCAGGCGAAC
CTGGAAAGAATGGAGCTAAGGGAGAGCCTGGCCCTAGAGGTGAGCGTGGTGAAGCT
GGAATCCCTGGTGTTCCAGGCGCCAAAGGCGAAGATGGTAAAGACGGAAGTCCAGG
TGAGCCAGGTGCCAACGGAGAACGAGGCTTTCCAGGCGAGAGGGGAGTTCAAGGCT
TTAGAGGTCCAGCCGGACCTAATGGCATACCTGGTGAGAAGGGACCAGCTGGTGAG
AGAGGTGCACCTGGTCCAGCCGGACCAAGAGGTGCTGCCGGAGAACCTGGCAGAGA
TGGAGTTCCAGGTGGCCCAGGTATGAGAGGTATGCCTGGATCACCAGGCGGTCCAG
GTTCTGATGGTAAGCCTGGTCCACCTGGATCTCAGGGAGAAAGTGGTAGACCAGGT
CCACCTGGACCATCCGGTCCAAGAGGTCAACCAGGTGTGATGGGTTTCCCTGGACCT
AAAGGCAACGACGGTGCACCTGGTAAGAACGGTGAAAGAGGTGGTCCAGGCGGAC
CTGGACCACAAGGTCCACCAGGCAAGAACGGAGAGACTGGACCACAGGGACCTCC
AGGACCAACCGGACCAGGCGGAGACAAAGGTGACACTGGTCCACCTGGACCTCAGG
GATTGCAGGGTTTACCAGGCACTGGCGGTCCACCTGGCGAGAACGGTAAACCTGGC
GAACCAGGCCCTAAGGGTGATGCTGGTGCACCTGGAGCCCCTGGTGGCAAAGGCGA
GAGAGGTTTCCCTGGAGAACGAGGTGTTCAGGGTTTAGCCGGAGCACCAGGCTTGA
GAGGCGGTGCCGGACCACCAGGTCCAGAGGGTGGTAAAGGCGCTGCCGGACCACCT
GGACCACCAGGCGCTGCTGGTACACCAGGCGAACGTGGATTTCCAGGTGAACGTGG
AGTACAAGGTAGTCCAGGTCCAAAGGGAGACAAGGGTGAACCTGGTGGCCCTGGTG
CTGATGGTGTGCCTGGCAAAGATGGTCCACGTGGTCCAACTGGACCTATTGGTCCAC
CTGGTCCAGCAGGTCAGCCTGGAGATAAGGGCGAAGGCGGTGCTCCAGGCTTGCCT
GGCATCGCTGGACCACGAGGTTCACCTGGAGAGAGAGGCGAAACGGGACCACCAG
GTCCAGCAGGCTTCCCTGGAGCACCTGGTCAGAATGGTGAACGAGGTTTCCCAGGTG
AGAGAGGTGTACAGGGAGAGAAGGGAGAGGGTGGACCTCCAGGCGTAGCTGGTCC
ACCTGGAGGTTCCGGACCAGCCGGTCCACCAGGTCCACAAGGTGTGAAAGGCGAAC
GAGGCTCACCTGGAGGCCCAGGTGCAGCTGGATTTCCAGGTGCTAGAGGCTTGCCA
GGTCCACCAGGCTCTAACGGAAACCCTGGTCCACCTGGACCTTCTGGCTCTCCAGGC
AAAGACGGACCTCCAGGTCCAGCAGGTAACACTGGCGCTCCAGGTTCACCAGGTGT
CTCTGGTCCAAAGGGTGACGCTGGCCAACCAGGCGAGAAGGGAAGTCCAGGCGCAC
AAGGCCCTCCAGGCGCACCTGGTCCATTAGGTATCGCAGGTATCACGGGTGCAAGA
GGTTTGGCTGGTCCACCTGGAATGCCTGGACCAAGAGGTTCTCCAGGTCCACAGGGA
GTTAAAGGCGAATCAGGTAAGCCAGGTGCAAACGGCTTATCTGGAGAAAGAGGTCC
ACCTGGTCCACAAGGCCTTCCAGGCCTTGCAGGTACAGCCGGAGAGCCAGGTAGAG
ATGGTAACCCTGGCTCAGATGGACTGCCAGGTAGGGATGGATCACCTGGCGGAAAG
GGCGACAGAGGTGAGAATGGCTCCCCTGGTGCACCTGGTGCACCTGGCCATCCAGG
ACCTCCAGGCCCTGTTGGCGAAAGAGGCTTCCCAGGCGAACGTGGTGTTCAAGGCC
CTGCCGGTCCAGCTGGCGCACCTGGACCAGCAGGCAGTAGAGGCGCACCTGGACCT
CAAGGCCCAAGAGGCGATAAGGGTGAAACTGGTGAGCGAGGCGCAGCTGGCATTA
AAGGCCATCGTGGCTTTCCAGGCAATCCAGGCGCTCCAGGCAGTCCAGGTCCAGCT
GGTCAACAAGGTGCTATTGGCTCACCAGGTCCAGCCGGACCTCGTGGACCTGTAGGT
CCATCTGGTCCACCAGGTAAAGATGGCACTTCAGGTCATCCAGGTCCAATCGGTCCA
CCTGGACCTAGAGGCAACAGAGGCGAGCGAGGTTCTGAAGGCTCTCCAGGTCACCC
TGGACAACCAGGTCCACCTGGTCCACCTGGCGCACCTGGACCATGTTGTGGAGGTGT
GGGAGCTGCTGCTATCGCTGGAATAGGCGGAGAGAAAGCTGGTGGTTTCGCACCTT
ATTACGGT.

The disclosure provides an expression vector, which includes the above nucleotide sequence. The expression vector can include regulatory sequences, such as start and stop codons for transcription and translation, the regulatory sequences have specificity for a type of the host of the vector to be introduced, such as bacteria, fungi, plants, or animals, depending on whether the vector is based on deoxyribonucleic acid (DNA) or ribonucleic acid (RNA). In a specific embodiment, the expression vector is pPIC9k, and the nucleotide sequence of the pPIC9k is as shown in SEQ ID NO: 3.

SEQ ID NO: 3 is shown as follows:

AGATCTAACATCCAAAGACGAAAGGTTGAATGAAACCTTTTTGCCATCCGAC
ATCCACAGGTCCATTCTCACACATAAGTGCCAAACGCAACAGGAGGGGATACACTA
GCAGCAGACCGTTGCAAACGCAGGACCTCCACTCCTCTTCTCCTCAACACCCACTTT
TGCCATCGAAAAACCAGCCCAGTTATTGGGCTTGATTGGAGCTCGCTCATTCCAATT
CCTTCTATTAGGCTACTAACACCATGACTTTATTAGCCTGTCTATCCTGGCCCCCCTG
GCGAGGTTCATGTTTGTTTATTTCCGAATGCAACAAGCTCCGCATTACACCCGAACA
TCACTCCAGATGAGGGCTTTCTGAGTGTGGGGTCAAATAGTTTCATGTTCCCCAAAT
GGCCCAAAACTGACAGTTTAAACGCTGTCTTGGAACCTAATATGACAAAAGCGTGA
TCTCATCCAAGATGAACTAAGTTTGGTTCGTTGAAATGCTAACGGCCAGTTGGTCAA
AAAGAAACTTCCAAAAGTCGCCATACCGTTTGTCTTGTTTGGTATTGATTGACGAAT
GCTCAAAAATAATCTCATTAATGCTTAGCGCAGTCTCTCTATCGCTTCTGAACCCCG
GTGCACCTGTGCCGAAACGCAAATGGGGAAACACCCGCTTTTTGGATGATTATGCAT
TGTCTCCACATTGTATGCTTCCAAGATTCTGGTGGGAATACTGCTGATAGCCTAACG
TTCATGATCAAAATTTAACTGTTCTAACCCCTACTTGACAGCAATATATAAACAGAA
GGAAGCTGCCCTGTCTTAAACCTTTTTTTTTATCATCATTATTAGCTTACTTTCATAAT
TGCGACTGGTTCCAATTGACAAGCTTTTGATTTTAACGACTTTTAACGACAACTTGA
GAAGATCAAAAAACAACTAATTATTCGAAGGATCCAAACGATGAGATTTCCTTCAA
TTTTTACTGCAGTTTTATTCGCAGCATCCTCCGCATTAGCTGCTCCAGTCAACACTAC
AACAGAAGATGAAACGGCACAAATTCCGGCTGAAGCTGTCATCGGTTACTCAGATT
TAGAAGGGGATTTCGATGTTGCTGTTTTGCCATTTTCCAACAGCACAAATAACGGGT
TATTGTTTATAAATACTACTATTGCCAGCATTGCTGCTAAAGAAGAAGGGGTATCTC
TCGAGAAAAGAGAGGCTGAAGCTTACGTAGAATTCCCTAGGGCGGCCGCGAATTAA
TTCGCCTTAGACATGACTGTTCCTCAGTTCAAGTTGGGCACTTACGAGAAGACCGGT
CTTGCTAGATTCTAATCAAGAGGATGTCAGAATGCCATTTGCCTGAGAGATGCAGGC
TTCATTTTTGATACTTTTTTATTTGTAACCTATATAGTATAGGATTTTTTTTGTCATTT
TGTTTCTTCTCGTACGAGCTTGCTCCTGATCAGCCTATCTCGCAGCTGATGAATATCT
TGTGGTAGGGGTTTGGGAAAATCATTCGAGTTTGATGTTTTTCTTGGTATTTCCCACT
CCTCTTCAGAGTACAGAAGATTAAGTGAGAAGTTCGTTTGTGCAAGCTTATCGATAA
GCTTTAATGCGGTAGTTTATCACAGTTAAATTGCTAACGCAGTCAGGCACCGTGTAT
GAAATCTAACAATGCGCTCATCGTCATCCTCGGCACCGTCACCCTGGATGCTGTAGG
CATAGGCTTGGTTATGCCGGTACTGCCGGGCCTCTTGCGGGATATCGTCCATTCCGA
CAGCATCGCCAGTCACTATGGCGTGCTGCTAGCGCTATATGCGTTGATGCAATTTCT
ATGCGCACCCGTTCTCGGAGCACTGTCCGACCGCTTTGGCCGCCGCCCAGTCCTGCT
CGCTTCGCTACTTGGAGCCACTATCGACTACGCGATCATGGCGACCACACCCGTCCT
GTGGATCTATCGAATCTAAATGTAAGTTAAAATCTCTAAATAATTAAATAAGTCCCA
GTTTCTCCATACGAACCTTAACAGCATTGCGGTGAGCATCTAGACCTTCAACAGCAG
CCAGATCCATCACTGCTTGGCCAATATGTTTCAGTCCCTCAGGAGTTACGTCTTGTGA
AGTGATGAACTTCTGGAAGGTTGCAGTGTTAACTCCGCTGTATTGACGGGCATATCC
GTACGTTGGCAAAGTGTGGTTGGTACCGGAGGAGTAATCTCCACAACTCTCTGGAGA
GTAGGCACCAACAAACACAGATCCAGCGTGTTGTACTTGATCAACATAAGAAGAAG
CATTCTCGATTTGCAGGATCAAGTGTTCAGGAGCGTACTGATTGGACATTTCCAAAG
CCTGCTCGTAGGTTGCAACCGATAGGGTTGTAGAGTGTGCAATACACTTGCGTACAA
TTTCAACCCTTGGCAACTGCACAGCTTGGTTGTGAACAGCATCTTCAATTCTGGCAA
GCTCCTTGTCTGTCATATCGACAGCCAACAGAATCACCTGGGAATCAATACCATGTT
CAGCTTGAGACAGAAGGTCTGAGGCAACGAAATCTGGATCAGCGTATTTATCAGCA
ATAACTAGAACTTCAGAAGGCCCAGCAGGCATGTCAATACTACACAGGGCTGATGT
GTCATTTTGAACCATCATCTTGGCAGCAGTAACGAACTGGTTTCCTGGACCAAATAT
TTTGTCACACTTAGGAACAGTTTCTGTTCCGTAAGCCATAGCAGCTACTGCCTGGGC
GCCTCCTGCTAGCACGATACACTTAGCACCAACCTTGTGGGCAACGTAGATGACTTC
TGGGGTAAGGGTACCATCCTTCTTAGGTGGAGATGCAAAAACAATTTCTTTGCAACC
AGCAACTTTGGCAGGAACACCCAGCATCAGGGAAGTGGAAGGCAGAATTGCGGTTC
CACCAGGAATATAGAGGCCAACTTTCTCAATAGGTCTTGCAAAACGAGAGCAGACT
ACACCAGGGCAAGTCTCAACTTGCAACGTCTCCGTTAGTTGAGCTTCATGGAATTTC
CTGACGTTATCTATAGAGAGATCAATGGCTCTCTTAACGTTATCTGGCAATTGCATA
AGTTCCTCTGGGAAAGGAGCTTCTAACACAGGTGTCTTCAAAGCGACTCCATCAAAC
TTGGCAGTTAGTTCTAAAAGGGCTTTGTCACCATTTTGACGAACATTGTCGACAATT
GGTTTGACTAATTCCATAATCTGTTCCGTTTTCTGGATAGGACGACGAAGGGCATCT
TCAATTTCTTGTGAGGAGGCCTTAGAAACGTCAATTTTGCACAATTCAATACGACCT
TCAGAAGGGACTTCTTTAGGTTTGGATTCTTCTTTAGGTTGTTCCTTGGTGTATCCTG
GCTTGGCATCTCCTTTCCTTCTAGTGACCTTTAGGGACTTCATATCCAGGTTTCTCTC
CACCTCGTCCAACGTCACACCGTACTTGGCACATCTAACTAATGCAAAATAAAATAA
GTCAGCACATTCCCAGGCTATATCTTCCTTGGATTTAGCTTCTGCAAGTTCATCAGCT
TCCTCCCTAATTTTAGCGTTCAACAAAACTTCGTCGTCAAATAACCGTTTGGTATAAG
AACCTTCTGGAGCATTGCTCTTACGATCCCACAAGGTGGCTTCCATGGCTCTAAGAC
CCTTTGATTGGCCAAAACAGGAAGTGCGTTCCAAGTGACAGAAACCAACACCTGTTT
GTTCAACCACAAATTTCAAGCAGTCTCCATCACAATCCAATTCGATACCCAGCAACT
TTTGAGTTGCTCCAGATGTAGCACCTTTATACCACAAACCGTGACGACGAGATTGGT
AGACTCCAGTTTGTGTCCTTATAGCCTCCGGAATAGACTTTTTGGACGAGTACACCA
GGCCCAACGAGTAATTAGAAGAGTCAGCCACCAAAGTAGTGAATAGACCATCGGGG
CGGTCAGTAGTCAAAGACGCCAACAAAATTTCACTGACAGGGAACTTTTTGACATCT
TCAGAAAGTTCGTATTCAGTAGTCAATTGCCGAGCATCAATAATGGGGATTATACCA
GAAGCAACAGTGGAAGTCACATCTACCAACTTTGCGGTCTCAGAAAAAGCATAAAC
AGTTCTACTACCGCCATTAGTGAAACTTTTCAAATCGCCCAGTGGAGAAGAAAAAG
GCACAGCGATACTAGCATTAGCGGGCAAGGATGCAACTTTATCAACCAGGGTCCTA
TAGATAACCCTAGCGCCTGGGATCATCCTTTGGACAACTCTTTCTGCCAAATCTAGG
TCCAAAATCACTTCATTGATACCATTATTGTACAACTTGAGCAAGTTGTCGATCAGC
TCCTCAAATTGGTCCTCTGTAACGGATGACTCAACTTGCACATTAACTTGAAGCTCA
GTCGATTGAGTGAACTTGATCAGGTTGTGCAGCTGGTCAGCAGCATAGGGAAACAC
GGCTTTTCCTACCAAACTCAAGGAATTATCAAACTCTGCAACACTTGCGTATGCAGG
TAGCAAGGGAAATGTCATACTTGAAGTCGGACAGTGAGTGTAGTCTTGAGAAATTCT
GAAGCCGTATTTTTATTATCAGTGAGTCAGTCATCAGGAGATCCTCTACGCCGGACG
CATCGTGGCCGACCTGCAGGGGGGGGGGGGGCGCTGAGGTCTGCCTCGTGAAGAAG
GTGTTGCTGACTCATACCAGGCCTGAATCGCCCCATCATCCAGCCAGAAAGTGAGG
GAGCCACGGTTGATGAGAGCTTTGTTGTAGGTGGACCAGTTGGTGATTTTGAACTTT
TGCTTTGCCACGGAACGGTCTGCGTTGTCGGGAAGATGCGTGATCTGATCCTTCAAC
TCAGCAAAAGTTCGATTTATTCAACAAAGCCGCCGTCCCGTCAAGTCAGCGTAATGC
TCTGCCAGTGTTACAACCAATTAACCAATTCTGATTAGAAAAACTCATCGAGCATCA
AATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCC
TGGTATCGGTCTGCGATTCCGACTCGTCCAACATCAATACAACCTATTAATTTCCCCT
CGTCAAAAATAAGGTTATCAAGTGAGAAATCACCATGAGTGACGACTGAATCCGGT
GAGAATGGCAAAAGCTTATGCATTTCTTTCCAGACTTGTTCAACAGGCCAGCCATTA
CGCTCGTCATCAAAATCACTCGCATCAACCAAACCGTTATTCATTCGTGATTGCGCC
TGAGCGAGACGAAATACGCGATCGCTGTTAAAAGGACAATTACAAACAGGAATCGA
ATGCAACCGGCGCAGGAACACTGCCAGCGCATCAACAATATTTTCACCTGAATCAG
GATATTCTTCTAATACCTGGAATGCTGTTTTCCCGGGGATCGCAGTGGTGAGTAACC
ATGCATCATCAGGAGTACGGATAAAATGCTTGATGGTCGGAAGAGGCATAAATTCC
GTCAGCCAGTTTAGTCTGACCATCTCATCTGTAACATCATTGGCAACGCTACCTTTGC
CATGTTTCAGAAACAACTCTGGCGCATCGGGCTTCCCATACAATCGATAGATTGTCG
CACCTGATTGCCCGACATTATCGCGAGCCCATTTATACCCATATAAATCAGCATCCA
TGTTGGAATTTAATCGCGGCCTCGAGCAAGACGTTTCCCGTTGAATATGGCTCATAA
CACCCCTTGTATTACTGTTTATGTAAGCAGACAGTTTTATTGTTCATGATGATATATT
TTTATCTTGTGCAATGTAACATCAGAGATTTTGAGACACAACGTGGCTTTCCCCCCCC
CCCCTGCAGGTCGGCATCACCGGCGCCACAGGTGCGGTTGCTGGCGCCTATATCGCC
GACATCACCGATGGGGAAGATCGGGCTCGCCACTTCGGGCTCATGAGCGCTTGTTTC
GGCGTGGGTATGGTGGCAGGCCCCGTGGCCGGGGGACTGTTGGGCGCCATCTCCTTG
CATGCACCATTCCTTGCGGCGGCGGTGCTCAACGGCCTCAACCTACTACTGGGCTGC
TTCCTAATGCAGGAGTCGCATAAGGGAGAGCGTCGAGTATCTATGATTGGAAGTAT
GGGAATGGTGATACCCGCATTCTTCAGTGTCTTGAGGTCTCCTATCAGATTATGCCC
AACTAAAGCAACCGGAGGAGGAGATTTCATGGTAAATTTCTCTGACTTTTGGTCATC
AGTAGACTCGAACTGTGAGACTATCTCGGTTATGACAGCAGAAATGTCCTTCTTGGA
GACAGTAAATGAAGTCCCACCAATAAAGAAATCCTTGTTATCAGGAACAAACTTCTT
GTTTCGAACTTTTTCGGTGCCTTGAACTATAAAATGTAGAGTGGATATGTCGGGTAG
GAATGGAGCGGGCAAATGCTTACCTTCTGGACCTTCAAGAGGTATGTAGGGTTTGTA
GATACTGATGCCAACTTCAGTGACAACGTTGCTATTTCGTTCAAACCATTCCGAATC
CAGAGAAATCAAAGTTGTTTGTCTACTATTGATCCAAGCCAGTGCGGTCTTGAAACT
GACAATAGTGTGCTCGTGTTTTGAGGTCATCTTTGTATGAATAAATCTAGTCTTTGAT
CTAAATAATCTTGACGAGCCAAGGCGATAAATACCCAAATCTAAAACTCTTTTAAAA
CGTTAAAAGGACAAGTATGTCTGCCTGTATTAAACCCCAAATCAGCTCGTAGTCTGA
TCCTCATCAACTTGAGGGGCACTATCTTGTTTTAGAGAAATTTGCGGAGATGCGATA
TCGAGAAAAAGGTACGCTGATTTTAAACGTGAAATTTATCTCAAGATCTCTGCCTCG
CGCGTTTCGGTGATGACGGTGAAAACCTCTGACACATGCAGCTCCCGGAGACGGTC
ACAGCTTGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCATAT
GCGGTGTGAAATACCGCACAGATGCGTAAGGAGAAAATACCGCATCAGGCGCTCTT
CCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGA
AAGAACATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGCT
CAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCC
GCCTTTCTCCCTTCGGGAAGCGTGGCGCTTTCTCAATGCTCACGCTGTAGGTATCTCA
GTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGAAGGAC
AGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACC
TATCTCAGCGATCTGTCTATTTCGTTCATCCATAGTTGCCTGACTCCCCGTCGTGTAG
ATAACTACGATACGGGAGGGCTTACCATCTGGCCCCAGTGCTGCAATGATACCGCG
AGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCA
TTGCTGCAGGCATCGTGGTGTCACGCTCGTCGTTTGGTATGGCTTCATTCAGCTCCGG
TTCCCAACGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTATCACTCATG
GTTATGGCAGCACTGCATAATTCTCTTACTGTCATGCCATCCGTAAGATGCTTTTCTG
TGACTGGTGAGTACTCAACCAAGTCATTCTGAGAATAGTGTATGCGGCGACCGAGTT
GCTCTTGCCCGGCGTCAACACGGGATAATACCGCGCCACATAGCAGAACTTTAAAA
GTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTA
CTTTCACCAGCGTTTCTGGGTGAGCAAAAACAGGAAGGCAAAATGCCGCAAAAAAG
GGAATAAGGGCGACACGGAAATGTTGAATACTCATACTCTTCCTTTTTCAATATTAT
TGAAGCATTTATCAGGGTTATTGTCTCATGAGCGGATACATATTTGAATGTATTTAG
AAAAATAAACAAATAGGGGTTCCGCGCACATTTCCCCGAAAAGTGCCACCTGACGT
CTAAGAAACCATTATTATCATGACATTAACCTATAAAAATAGGCGTATCACGAGGCC
CTTTCGTCTTCAAGAATTAATTCTCATGTTTGACAGCTTATCATCGATAAGCTGACTC
ATGTTGGTATTGTGAAATAGACGCAGATCGGGAACACTGAAAAATAACAGTTATTA
TTCG.

The disclosure provides a host cell, and the host cell includes the above nucleotide sequence. The host cell refers to cells having been introduced with exogenous nucleic acid, including the offspring of such cells. The host cell includes transformants and transformed cells, including primary transformed cells and their offspring, regardless of the number of passages. The offspring may not have exactly the same nucleic acid content as parental cells, but they may contain mutations.

In a specific embodiment, the host cell is any one selected from the group consisting of Pichia pastoris, Saccharomyces cerevisiae, Escherichia coli, and Bacillus subtilis.

In a specific embodiment, the host cell is Pichia pastoris GS115.

The disclosure provides a method for preparing any one of the aforementioned recombinant human type III collagen, and the method includes the following steps.

The recombinant human type III collagen is expressed by utilizing the host cell to obtain an expressed host cell, and the expressed host cell is separated to obtain separated collagen, and the separated collagen is purified to obtain purified collagen (i.e., the recombinant human type III collagen).

The recombinant human type III collagen expressed by utilizing the host cell refers to cultivate the host cell, and the culture medium and culture conditions are well-known to those skilled in the art.

In a specific embodiment, the host cell is the Pichia pastoris. After obtaining the Pichia pastoris genetically engineered bacteria, the specific culture conditions are as follows. The Pichia pastoris genetically engineered bacteria are inoculated in a yeast extract-peptone-dextrose (YPD) culture medium, and are cultivated under 220 revolutions per minute (rpm) at 30 Celsius degrees (° C.) for 22 hours (h) to 24 h, to an optical density at 600 nanometers (nm) (OD₆₀₀) being 18 to 22, to thereby obtain seed fermentation broth. After the seed fermentation broth is expanded by culture, it is inoculated into a NBS 415 fermentor with an initial volume of 5 liters (L) according to the inoculation amount of 10%. The culture temperature is 28° C. to 30° C., the power of hydrogen (pH) is 5.0 to 6.0, and the dissolved oxygen is controlled at 20% to 30%. When the glycerol is exhausted, glycerol fed-batch culture is started, and when a wet weight of bacteria reaches more than 180 grams per liter (g/L), induction culture is started.

For the expression mode, the disclosure does not limit it in any way, and it can be confirmed as needed, for example, the expression is induction expression, and the inducer is methanol for the induction expression.

In a specific embodiment, methanol feeding for induction is performed. During the induction stage, a temperature is 28° C., a pH is 5.0, the induction is performed for 48 h before the fermentation is harvested.

The disclosure does not limit the separation and purification method, which can be determined according to, for example, salting out method, ultrafiltration method, affinity chromatography method and gel filtration chromatography method.

The disclosure further provides the aforementioned recombinant human type III collagen, the recombinant human type III collagen encoded by the aforementioned gene, the recombinant human type III collagen expressed by the aforementioned expression vector, or the recombinant human type III collagen produced by the aforementioned host cell, which has the ability to promote angiogenesis and wound healing.

### Embodiment 1: Expression of the Recombinant Human Type III Collagen

The gene encoding the recombinant human type III collagen of the disclosure is chemically synthetized, the nucleotide sequence of the gene is as shown in SEQ ID NO: 2. During synthesis, EcoR I and Not I recognition sites, as well as signal peptide recognition sites are added at the 5′ and 3′ ends of the gene to obtain synthetized gene. The synthetized gene is linearized by a restriction enzyme Sac I to obtain linearized gene, and the linearized gene is cloned into the expression vector pPIC9K to thereby obtain pPIC9K-ROL (III) cloning plasmid. The Pichia pastoris GS115 is used as the expression host bacteria, the obtained pPIC9K-ROL (III) cloning plasmid is linearized and then transformed into the Pichia pastoris GS115 by electroporation, to thereby obtain GS115-pPIC9K-ROL (III). The GS115-pPIC9K-ROL (III) is cultivated at 30° C. for 72 h to obtain the Pichia pastoris genetically engineered bacteria.

The obtained Pichia pastoris genetically engineered bacteria is inoculated into the YPD culture medium. When the Pichia pastoris genetically engineered bacteria is cultivated to OD₆₀₀ of 19.88, the cultivated Pichia pastoris genetically engineered bacteria is inoculated into the NBS 415 fermentor with an initial volume of 5 L according to the inoculation amount of 10%, the culture temperature is 30° C., pH is 5.5, and the dissolved oxygen is controlled at 20%. When the glycerol is exhausted, glycerol fed-batch culture is started, and when a wet weight of Pichia pastoris genetically engineered bacteria reaches more than 190 g/L, methanol is added at a flow rate of 80 milliliter per hour (mL/h) for induction culture. During the induction stage, the temperature is 28° C., and the pH is 5.0, and the induction is performed for 48 h to obtain induced Pichia pastoris genetically engineered bacteria before fermentation is harvested. The induced Pichia pastoris genetically engineered bacteria is centrifugated to collect supernatant.

### Embodiment 2: Purification of the Recombinant Human Type III Collagen

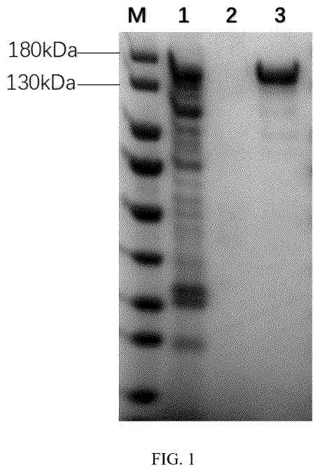

1. The supernatant collected by centrifugation is ultrafiltered to 50% of the initial volume, 5 times the volume of pure water is added into the ultrafiltered supernatant to obtain a diluted supernatant, and then the diluted supernatant is concentrated by ultrafiltration to 5% of the initial volume to obtain concentrated supernatant.
2. The concentrated supernatant is added with saturated ammonium sulfate to obtain a mixed solution, and an added amount of the saturated ammonium sulfate is 60% of the volume of the concentrated supernatant. The mixed solution is stirred at the room temperature for 30 minutes (min) to obtain a stirred solution. The stirred solution is centrifugated at 9000 rpm for 10 min to collect sediment. The sediment is dissolved in 500 mL phosphate buffered saline (PBS) with a molar mass of 0.05 moles per liter (M) and a pH of 7.0 to obtain a mixture. The mixture is filtered by a 0.22 microns filter membrane to obtain a PBS protein solution.
3. A balanced buffer solution is prepared according to an isoelectric point of the protein (i.e., the recombinant human type III collagen): a sodium phosphate buffer solution with a molar mass of 20 millimoles per liter (mmol/L) and a pH of 6.0 is recorded as a solution A; and an eluent with a pH of 6.0 prepared by mixing sodium phosphate with a molar mass of 20 mmol/L and sodium chloride (NaCl) with a molar mass of 1.0 mole per liter (mol/L) is recorded as a solution B. The PBS protein solution obtained by the previous step is diluted with the solution A at a ratio of 10:1 to prepare a sample solution. The sample solution is filtered and then loaded onto a 25 mL CM-Sepharose cation exchange chromatography column. Before loading, the column is balanced with the balanced buffer solution. After loading, the CM-Sepharose cation exchange chromatography column is washed by using the solution A for two column volumes, and then is subjected to gradient elution by using 70% solution A and 30% solution B with a flow rate of 2 mL/min. Each eluted component is collected and detected by using sodium dodecyl sulfate polyacrylamide gel electrophoresis (SDA-PAGE). The detected results are shown in FIG. 1, where M represents marker, lane 1 is the sample solution, lane 2 is the sample effluent, and lane 3 is the purified protein, namely recombinant GS115-pPIC9K-ROL (III) protein (i.e., the recombinant human type III collagen).
4. A Sephadex 200 gel column is selected to further purify the target protein (i.e., the recombinant human type III collagen) according to a distribution range of protein molecular weight obtained after ion exchange chromatography. An operation process of automated knowledge transfer and analysis (AKTA) is as follows: the balanced buffer solution, that is, 0.01 mol/L PBS and 0.05 mol/L NaCl are used to wash the Sephadex 200 gel column until the baseline is stable; then the recombinant GS115-pPIC9K ROL (III) protein component eluted by the ion exchange column obtained in the previous step is loaded onto the gel filtration chromatography column filled with a gel filter filler such as Superdex 200. The gel filtration chromatography column is eluted by the eluent with a flow rate of 10 mL/min, and an ultraviolet detection wavelength is 215 nm. Finally, the target protein component is collected after SDS-PAGE electrophoresis detection.
5. Ultrafiltration desalting: the target protein component is desalted by a G25 desalination column, that is, using 25 mL G25 filler. The operation process of the ultrafiltration desalting is similar with that of the gel filtration chromatography, each sample loading is 6.5 mL, and approximately 8 mL is collected. The desalting process is completed after 10 min of sample loading to obtain desalted protein.
6. The desalted protein is concentrated by ultrafiltration to 30% of the initial volume of the desalted protein to obtain concentrated protein. The concentrated protein is pre-frozen in a refrigerator at −20° C. for 4 h to obtain pre-frozen protein. The pre-frozen protein is freeze-dried in a vacuum freeze dryer for 48 h to obtain freeze-dried protein. The freeze-dried protein is stored into a refrigerator at −80° C. for later use.

### Embodiment 3: Application of the Recombinant Human Type III Collagen

### 1. Cell Proliferation Experiment

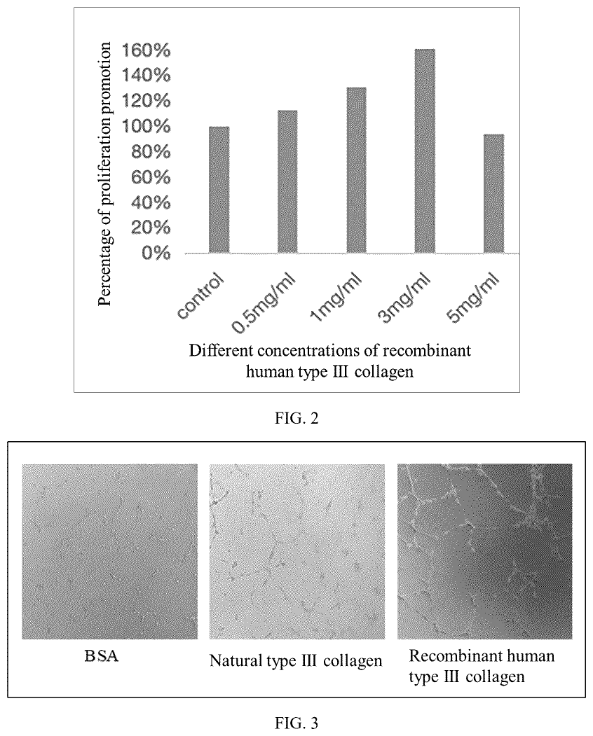

An in vitro cell proliferation experimental model is used to evaluate cell activity induced by the recombinant GS115-pPIC9K-ROL (III) protein. In short, human umbilical vein endothelial cells (HUVEC) in a logarithmic growth phase are inoculated to a 96-well tissue culture plate, and the 96-well tissue culture plate inoculated with HUVEC cells is cultivated for 1 day (d) in a biochemical incubator with a volume fraction of 5% carbon dioxide (CO₂) at 37° C. to obtain cultivated HUVEC cells. The purified recombinant GS115-pPIC9K-ROL (III) protein is dissolved in a Dulbecco's modified Eagle medium (DMEM), and prepared to standard extracts with concentrations of 0.5 milligrams per milliliter (mg/mL), 1 mg/mL, 3 mg/mL and 5 mg/ml respectively. The standard extracts are filtered and sterilized, and then added into the cultivated HUVEC cells to be incubated for 24 h, then the old culture medium (i.e., the DMEM medium) is discarded. In a control group, 100 microliters (μL) of fresh complete culture medium is added. In experimental groups (i.e., the standard extracts with concentrations of 0.5 mg/mL, 1 mg/mL, 3 mg/mL and 5 mg/mL), 100 μL of recombinant GS115-pPIC9K-ROL (III) protein solution with equal volume to the fresh complete culture medium is added into each experimental group. Cell proliferation in the control group and the experimental groups is verified by using a cell counting kit-8 (CCK8) method, and absorbance is measured at 450 nm. The cell proliferation rates of the control group and the experimental groups are shown in FIG. 2.

### 2. Angiogenesis Promotion Experiment

### Preparation of Basement Membrane Matrix

The day before the experiment, the basement membrane matrix stored at −20° C. is buried in crushed ice and placed in a refrigerator at 4° C., allowing the basement membrane matrix to slowly melt overnight to obtain melted basement membrane matrix. Before the experiment begins, the melted basement membrane matrix is placed in an ice box, a 48-well plate and 1 mL pipette tip are pre-cooled on ice to obtain pre-cooled 48-well plate and pre-cooled gun head, and the melted basement membrane matrix is mixed evenly with the pre-cooled pipette tip to obtain even basement membrane matrix.

### Laying of Basement Membrane Matrix

A pre-cooled 1.5 mL centrifuge tube is prepared for diluting the even basement membrane matrix with a 1:1 volume ratio of the even basement membrane matrix to a DMEM medium to obtain diluted basement membrane matrix. After dilution, 80 μL of the diluted basement membrane matrix is added to each well of the pre-cooled 48-well plate vertically, to thereby to avoid the formation of bubbles. The pre-cooled 48-well plate added with the diluted basement membrane matrix is placed in a 37° C. incubator for 1 h to solidify the basement membrane matrix.

### Laying of Cells

Fifth generation HUVEC cells in good condition are used and digested to obtain digested HUVEC cells, and the digested HUVEC cells are resuspended in a DMEM medium containing 10% serum to obtain a resuspension solution, and the HUVEC cells in the resuspension solution are counted. 100 μL of the resuspension solution of approximately 50,000 HUVEC cells is added to each well of the pre-cooled 48-well plate added with the solidified basement membrane matrix, with three replicates per group. BSA, natural type III collagen, and purified recombinant GS115-pPIC9K-ROL (III) protein are sequentially dissolved in the DMEM medium at final concentrations of 3 mg/mL, and are respectively recorded as a negative control group, a positive control group, and an experimental group. 200 μL of each group is added into each well of the 48-well plate added with the resuspension solution. The 48-well plate added with each group is placed in a 37° C. incubator for cultivating for 12 h, and then angiogenesis is observed.

### Quantitative Analysis

Quantitative analysis is performed on the vascular network by using an Angiogenesis Analyzer plugin in an Image J software. The statistics of angiogenesis rates of each group are shown in Table 1, and the angiogenesis diagram is shown in FIG. 3.

TABLE 1
Statistics of angiogenesis rates

| | Negative control | Positive control | Experimental |
|---|---|---|---|
| | group | group | group |
| Angiogenesis rate | 100% | 105% | 142% |

The results show that the recombinant human type III collagen of the disclosure can significantly promote angiogenesis of the HUVEC cells, with a higher ability to promote angiogenesis than natural collagen and significantly higher than BSA. The recombinant human type III collagen of the disclosure has good cell compatibility and exhibits a good promoting effect, which is consistent with the results observed under light microscopy.

The various technical features of the above embodiments can be combined in any way. In order to make the description concise, not all possible combinations of the various technical features in the above embodiments have been described. However, as long as there is no contradiction in the combination of these technical features, they should be considered within the scope of this specification.

The above embodiments merely express some of the embodiments of the disclosure, and their descriptions are more specific and detailed, but should not be understood as limiting the scope of the disclosure. It should be pointed out that for those skilled in the art, multiple modifications and improvements can be made without departing from the inventive concept, which are within the scope of protection of the disclosure.

What is claimed is:

1. Recombinant human type III collagen for promoting angiogenesis, wherein the amino acid sequence of the recombinant human type III collagen is as shown in SEQ ID NO: 1.

2. A gene encoding the recombinant human type III collagen as claimed in claim 1, wherein the nucleotide sequence of the gene is as shown in SEQ ID NO: 2.

3. An application method of the recombinant human type III collagen as claimed in claim 1, comprising:
preparing a cardiovascular stent material or an artificial blood vessel by using the recombinant human type III collagen.

4. An application method of the recombinant human type III collagen as claimed in claim 1, comprising:
preparing a skin tissue repair material by using the recombinant human type III collagen.

5. An application method of the recombinant human type III collagen as claimed in claim 1, comprising:
preparing a preparation for treating cardiovascular diseases by using the recombinant human type III collagen.

6. An expression vector, comprising the gene as claimed in claim 2.

7. A host cell, comprising the expression vector as claimed in claim 6.

8. The host cell as claimed in claim 7, wherein the host cell is any one selected from the group consisting of Pichia pastoris, Saccharomyces cerevisiae, Escherichia coli, and Bacillus subtilis.

9. A method for preparing the recombinant human type III collagen by using the host cell as claimed in claim 7, comprising:
cultivating the host cell in a culture medium, and inducing expression of the recombinant human type III collagen to obtain an induced product, and purifying the induced product to obtain the recombinant human type III collagen.

10. The method as claimed in claim 9, wherein a method for the purifying is any one selected from the group consisting of a salting-out method, an ultrafiltration method, an affinity chromatography method and a gel filtration chromatography method.
